(19) United States
(12) Patent Application Publication
(10) Pub. No.: US 2024/0091741 A1
(43) Pub. Date: Mar. 21, 2024

(54) COMPOSITIONS AND METHODS FOR FILTERING MICROORGANISMS FROM A FLUID

(71) Applicants: George Mason University, Fairfax, VA (US); Food and Drug Administration, Silver Spring, MD (US)

(72) Inventors: Alessandra Luchini Kunkel, Burke, VA (US); Silvia Isabel Cachaco, Manassas, VA (US); Rocío Solange Prisby, Vienna, VA (US); Amanda Haymond Still, Manassas, VA (US); Lance Liotta, Bethesda, MD (US); Luisa Gregori, Bethesda, MD (US); Cyrus Bett, Damascus, MD (US)

(21) Appl. No.: 18/354,345

(22) Filed: Jul. 18, 2023

Related U.S. Application Data
(60) Provisional application No. 63/376,203, filed on Sep. 19, 2022.

(51) Int. Cl. B01J 20/26; A61L 15/26; A61L 15/56; B01D 15/38; C02F 1/28

(52) U.S. Cl. CPC B01J 20/265; A61L 15/26; A61L 15/56; B01D 15/3804; C02F 1/285

(57) ABSTRACT

The present disclosure relates to compositions, devices and methods for removing/filtering out microorganisms from a given fluid. The composition of the present invention comprises a non-water imbibing, biocompatible filament and at least one affinity ligand that is capable of capturing the microorganism(s). The invention also discloses methods for detection, diagnosis, and/or treatment.

### CROSS-REFERENCE TO RELATED APPLICATIONS

The present application claims priority under 35 U.S.C. § 119(e) to U.S. Provisional Application No. 63/376,203, filed Sep. 19, 2022, which is hereby incorporated by reference herein in its entirety.

### GOVERNMENT INTERESTS

This invention was made with government support under grant numbers AI154295, HD097472 awarded by the National Institutes of Health. The government has certain rights in the invention.

### FIELD OF THE INVENTION

The present invention relates to compositions, and methodologies including, but not limited to, for filtering microorganisms from different types of fluids.

### BACKGROUND

Currently available pathogen reduction technologies (PRTs) for purifying fluids by removing contaminates such as various microbes, including but not limited to pathogens, are not effective in completely eliminating the microbes/pathogens satisfactorily. As an example, blood and blood product purification from pathogen is an unresolved issue and therefore, transfusion of transmitted infections still occurs despite donor screening and blood testing. Additionally, current microorganism removal technologies are not applicable to whole blood and red blood cells and can further adversely affect platelet integrity. Several emerging pathogens pose a threat of contaminating the blood used for transfusion leading to severe health consequences for the recipient. Hence, in order to avoid the risk of transfusing microorganism-contaminated blood to a recipient, there is an unmet need in the art to develop compositions and methods that can reliably separate microbial contaminants from fluids such as blood. The present invention addressed this unmet need.

### SUMMARY OF THE INVENTION

In one aspect, the invention is related to a composition for separating microorganisms from various types of biological or environmental fluids. In certain embodiments, the composition comprises a non-water imbibing, biocompatible filament and at least one affinity ligand.

In certain embodiments, the filament is functionalized with the at least one affinity ligand. In certain embodiments, the at least one affinity ligand is a dye. In certain embodiments, the filament is a nylon filament.

In another aspect, the invention provides an affinity matrix comprising the composition as described elsewhere herein, where the composition comprises a non-water imbibing, biocompatible filament and at least one affinity ligand.

In yet another aspect, the invention comprises a filtration device comprising a column and the affinity matrix as described elsewhere herein.

In yet another aspect, the invention comprises a method for filtering a fluid, wherein the method comprises contacting the fluid with the affinity matrix of the filtration device described elsewhere herein, wherein a microorganism, if present in the fluid, is captured by the at least one affinity ligand.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a representative schematic showing filtration of fluids using pathogen reduction technology (PRT) affinity net device according to one embodiment of the present invention. Unwoven cloud of dyed nylon filaments are packed into a column. Blood is flown into the column, where pathogens are captured and retained by the affinity net or cloud of the nylon filaments.

FIG. 2 shows schematic representation of the methodology used to apply affinity ligands into the filaments.

FIG. 3 shows the appearance of filaments with the affinity networks bound to them.

FIG. 4 shows a schematic of representation of methodology used to assess yield of affinity ligands bound to the filament.

FIG. 5 shows 96 well plates with the different calibration curve dyes and nylon dissolved in solvent with hydrochloric acid.

FIG. 6 is a representation of the dot-blotting methodology used with lipopolysaccharide (LPS) and lipoteichoic acid (LTA).

FIG. 7 is a representation of methodology used to test bacteria captured by affinity network in the filaments.

FIG. 8 shows unwoven filaments used for bacterial capture.

FIG. 9 shows unfiltered (comprising filtered fluid) and filtered (comprising unfiltered fluid) plates.

FIG. 10 shows ligands selected for screen of bacterial capture.

FIG. 11 shows FT-IR spectrum of washed and autoclaved nylon. Spectrum was obtained using Agilent Cary 630 FTIR spectrometer. Conditions were as follows: sample scans: 32, background scans: 32, range: 4000-650 (cm⁻¹). Compared with commercially available Hummel Polymer sample.

FIG. 12 shows FT-IR spectrum of washed and autoclaved nylon and nylon dyed with dye 7 (blue-Methylene Blue). Spectrum was obtained using Agilent Cary 630 FTIR spectrometer. Conditions were as follows: sample scans: 32, background scans: 32, range: 4000-650 (cm⁻¹). In the circle is the area with peak intensity change

FIG. 13 top left shows comparison of absorbance between nylon and nylon treated with acetic acid. Top right shows comparison of absorbance between nylon and nylon bound with dye 7. Bottom shows comparison of absorbance between dye 7 and nylon bound to dye 7.

FIG. 14 shows average percentage of dye weight compared to the nylon weight.

FIGS. 15A-15B show dot-blot assay showing the initial solution (positive control, PC), and intensity of signal of glycocalyx present in the supernatant. A negative control (NC) was deposited in each membrane to assess the background signal. Each double dot represents one affinity network type. FIG. 15A is relative to lipoteichoic acid (LTA), FIG. 15B is relative to lipopolysaccharide (LPS).

FIG. 16 shows a photograph after incubating bacteria (E. coli) with the filament bound to the affinity networks (here dye 6 (safranin O)). After plating the nylon and the supernatant on the same plate, growth coming from the nylon filaments was observed.

FIG. 17 shows western blot SDS PAGE developed of plasma incubated with nylon (9-12) in comparison with plasma prior to incubation (2-5).

FIG. 18 illustrates optimal length of filament in PBS seen at 80 milligrams, with a comparison between top dyes (8, 9, 16, and 20), all dyes mixed, and undyed nylon.

FIG. 19 shows percentage of unbound bacteria in concentrations of 10 and 100 CFU/mL, with different ligands bound to the filament. This is the E. coli collection.

FIG. 20 shows percentage of unbound bacteria in concentrations of 10 and 100 CFU/mL, with different ligands bound to the filament. This is the S. epidermidis.

FIG. 21 illustrates bacterial glycoconjugates with specific differences between the types of bacteria. Common glycoconjugates in Gram-negative species are lipopolysaccharide (LPS) and lipooligosaccharide (LOS) structures, and common conjugates in Gram-positive species are wall teichoic acid and lipoteichoic acid (LTA). Both can have glycosylated flagella, pili or fimbriae, capsular polysaccharide, exopolysaccharide, and glycoproteins. Their cell walls contain a peptidoglycan layer.

FIG. 22 shows nylon fiber dye incorporation by different chemical class of dyes.

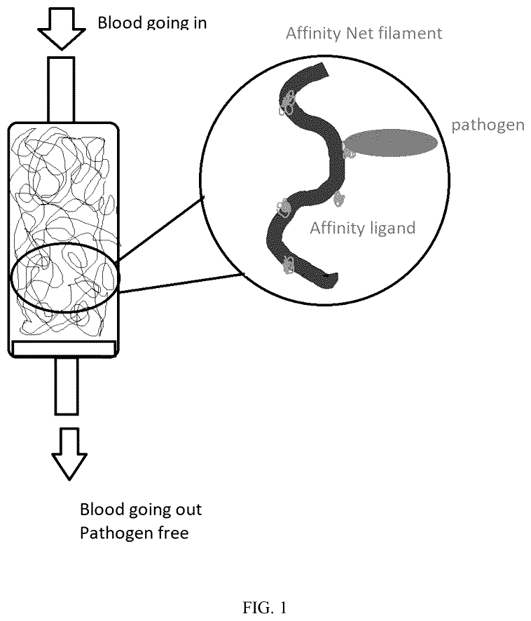

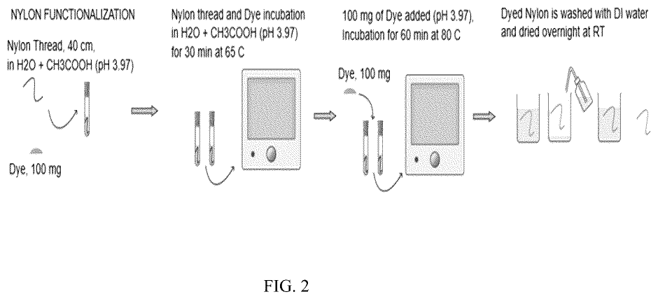

### DETAILED DESCRIPTION OF THE INVENTION

In one aspect, the present invention is related to removing microorganisms (e.g., pathogenic microorganisms) from transfusion products using a filtration device containing specific ligands to capture bacteria from blood. This approach is expected to have minimal impact on the treated blood because no compounds are added, and the blood is minimally manipulated. In some embodiments, different affinity ligands capture and remove microorganisms (e.g., bacteria) from the blood using filtration devices. In other embodiments, the present invention provides methods to reduce contamination in whole blood that can be effective against different categories of contaminants including, but not limited to, viruses, bacteria, fungi, and prions.

### Definition

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “amino acid” as used herein refers to natural amino acids, unnatural amino acids, and amino acid analogs, all in their various stereoisomers (e.g., D and L stereoisomers or other allostereomers if their structures so allow). Natural (or “naturally-occurring”) amino acids include the 20 “standard” amino acids that are encoded by the codons of the universal genetic code (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), as well as other “non-standard” amino acids that occur naturally but are not encoded by the codons of the universal genetic code (e.g., hydroxyproline, selenomethionine, and norleucine). Amino acids that are non-standard and/or non-naturally occurring include, without limitation, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid. 2-aminoisobutyric acid, 3-aminoisobutyric acid, 2-aminopimelic acid, 2,4-diaminoisobutyric acid, desmosine, 2,2′-diaminopimelic acid, 2,3-diaminopropionic acid, N-ethylglycine, N-ethylasparagine, hydroxylysine, allo-hydroxylysine, 3-hydroxyproline, 4-hydroxyproline, isodemosine, allo-isoleucine, N-methylglycine, N-methylisoleucine, N-methylvaline, norvaline, norleucine, ornithine, and pipecolic acid.

The term “affinity network” as used herein refers to the network formed when ligands are bound to the filaments.

The term “ligand” comprises compound that binds to any region(s) of a biomolecule present on the surface of a microorganism. In one embodiment, the ligand binds with a high affinity such that the dissociation constant is equal to or less than in nM range.

The term “affinity net device” or “device” as used herein are interchangeable and generally describe a device comprising affinity network.

As used herein, the term “subject” refers to a mammal, including, but not limited to, a human or non-human mammal, such as a bovine, equine, canine, ovine, feline, mouse, or monkey. The term “subject” may refer to an animal, which is the object of treatment, observation, or experiment (e.g., a patient).

In the methods described herein, the steps can be carried out in any order, except when a temporal or operational sequence is explicitly recited. Furthermore, specified steps can be carried out concurrently unless explicit claim language recites that they be carried out separately. For example, in some embodiments, a step of doing X and a step of doing Y can be conducted simultaneously within a single operation, and the resulting process will fall within the literal scope of the claimed process.

As used herein, the term “therapeutically effective amount” is an amount of a compound of the disclosure, that when administered to a patient, treats, minimizes, and/or ameliorates a symptom of the disease or disorder. The amount of a compound of the disclosure that constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

As used herein, the term “treatment” or “treating” is defined as the application or administration of a therapeutic agent, i.e., a compound useful within the disclosure (alone or in combination with another pharmaceutical agent), to a subject, or application or administration of a therapeutic agent to an isolated tissue or cell line from a subject (e.g., for diagnosis or ex vivo applications), who has cancer, a symptom of cancer or the potential to develop cancer, with the purpose to cure, heal, alleviate, relieve, alter, remedy, ameliorate, improve or affect cancer, the symptoms of cancer or the potential to develop cancer. Such treatments may be specifically tailored or modified, based on knowledge obtained from the field of pharmacogenomics.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

### Composition

In one aspect, the present invention provides a composition comprising a non-water biocompatible filament; and at least one affinity ligand. In certain embodiments, the filament is a non-woven filament. The filament is functionalized with the at least one affinity ligand.

In some embodiments, the composition is useful for separating microorganisms that may be present in a fluid. In one embodiment, the composition comprises non-woven, cloud-like filaments that are non-water imbibing and whose surface has been engineered for molecular recognition. In another embodiment, the molecular recognition is mediated by affinity ligands that are tightly bound to the filaments and establish strong chemical bonds with molecules decorating the surface of microorganisms including, but not limited to, bacteria, viruses, fungi, and protozoa.

In certain embodiments, the at least one affinity ligand is a dye. In certain embodiments, the dye is at least one selected from the group consisting of a direct dye, a sulfur dye, a reactive dye, a fast dye, an insoluble azoic dye, a phthalocyanine dye, an acid dye, a metal-complex dye, a basic dye, a metallic dye, a hydrophobic dye, an uncharged polar dye, and a disperse dye.

In certain embodiments, the dye is selected from the group consisting of Acid Red 87, Acid Red 92, Acid Orange 50, Acid Fuchsin, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Reactive Blue 21, Sudan I, Sudan IV, Sudan Black B, Oil Red O, Acid Black 48, Bismarck Brown Y, Alizarin Cyanin, and Eosin B.

In certain embodiments, the filament is a synthetic polymer selected from the group consisting of polytetrahydrofuran, polycaprolactone, polyvinylchloride, polyethylene, polypropylene, polytetrafluoroethylene, polymethylmethacrylate, dimethylaminododecyl methacrylate, trimethylcarbonate, and polyvinylidene fluoride. In certain embodiments, the filament is a polyamide filament. In certain embodiments, the filament is a nylon filament. In certain embodiments, the nylon filament is at least one selected from the group consisting of nylon 6; nylon 6,6; nylon 4,6; nylon 6,9; nylon 6,10; nylon 6,12; nylon 11; and nylon 12. In other embodiments, the filament is a natural polymer selected from the group consisting of silk, spider silk, cotton, and wool.

In other embodiments, the filament (which is cloud-like and non-woven) has rheological advantages e.g., does not cause sheer stress that damages the cellular content of the blood; it is biocompatible, does not cause cytotoxicity and does not leach toxic substances.

In certain embodiments, the at least one affinity ligand has an affinity for a biomolecule at a surface of a microorganism. In certain embodiments, the biomolecule is at least one selected from the group consisting of a protein, a glycan, a glycoprotein, a lipid, and a nucleic acid.

In certain embodiments, the microorganism is selected, for example, from the group consisting of a bacterium, a virus, a fungus, and a protozoan. In other embodiments, the microorganism is a pathogen. In another embodiment, the microorganism is a bacterium. In certain embodiments, the microorganism is selected from a group consisting of E. coli, B. cereus, Y enterocolitica, S. epidermidis, S. aureus, P. aeruginosa, S. marcescens, C. acnes, Streptococcus pneumoniae, Streptococcus bovis, Treponema pallidum, Brucella sp., Leptospira interrogans, Serratia sp., Enterobacter sp., Salmonella sp., Listeria monocytogenes, Shigella spp, Neisseria meningitidis, and Neisseria gonorrheae.

The at least one affinity ligand is easy to use, store and manipulate; and has a binding affinity strong enough to bind and separate the microorganism from a fluid.

In certain embodiments, an affinity ligand that targets a protein binds proportionally to protein's size. In certain embodiments, ligands have preferential affinity for certain amino acids. In certain embodiments, the larger the hydrophobic area of the ligand, the higher the binding affinity is. In certain embodiments, a metal containing ligands (such as, for example, copper containing dyes) have sufficient affinity towards certain biomolecules such as, for example, glycans.

In another aspect, the invention provides an affinity matrix comprising the composition described herein.

### Device

In another aspect, the present invention provides a filtration device comprising a column, and the affinity matrix as described elsewhere herein.

In certain embodiments, the matrix is contained inside the column. In certain embodiments, the matrix is attached to the column. In certain other embodiments, the matrix is not attached to the column.

In certain embodiments, the filtration device is used to separate/filter out microorganisms from various biological and environmental fluids listed elsewhere herein.

### Methods

In yet another aspect, the invention provides a method for filtering a fluid by removing a microorganism, the method comprising contacting the fluid with the affinity matrix of the filtration device as described elsewhere herein, wherein the microorganism, if present in the fluid, is captured by the affinity ligand(s).

In certain embodiments, the microbial population comprises at least one selected from the group consisting of a bacterium, a virus, a fungus, and a protozoan.

In other embodiments, the microorganism is a pathogen. In another embodiment, the microorganism is a bacterium.

In certain embodiment, the microorganism is selected, for example, from a group consisting of E. coli, B. cereus, Y. enterocolitica, S. epidermidis, S. aureus, P. aeruginosa, S. marcescens, C. acnes, Streptococcus pneumoniae, Streptococcus bovis, Treponema palladium, Brucella sp., Leptospira interrogans, Serratia sp., Enterobacter sp., Salmonella sp., Listeria monocytogenes, Shigella spp, Neisseria meningitidis, and Neisseria gonorrheae.

Using filaments with ligands as filtration devices is possible for many different products and processes that can be highly important, not only for a health application, but also for environmental ones. For example, in some embodiments, the filaments and methods of the invention can be applied not only toward removing pathogens from water but also pollutants and undesired products. In other embodiments, it could also have many agricultural or farming applications or be used for the filtration of water. In some embodiments, it is used in a method for filtrating milk or other animal products that will later be used for consumption. In other embodiments, as a filtration device, the present invention provides compositions and methods that improve lives, industry, and/or research.

In certain embodiments, the fluid is a biological fluid. In certain other embodiments, the fluid is an environmental fluid. In certain embodiments, the fluid is for example a blood, a serum, a cerebrospinal fluid, a vaginal fluid, a semen, plasma, platelets, and any other fraction of blood. In certain embodiments, the fluid is for example water, a plant lymph, and a sewerage. In certain embodiments, the water is, for example, food processing water, pond water, municipal water, soil percolating water, plant lymph, or sewerage.

Another the filaments with ligands of the present invention can be used are as detection devices, for example applied toward detecting different types of microorganisms. For example, chronic urinary tract infections are one of the major concerns for hospitals and patients whose quality of life is immensely impacted by urinary tract infections. In some embodiment, the filaments with ligands of the invention can be used to capture bacteria (e.g., a bacterium that cause UTI, such as e.g., gastrointestinal (GI) tract bacteria (e.g., E. coli)), which can then be plated and identified, thereby detecting a disease. In other embodiment, identification of the organism is carried out using e.g., PCR or Mass spectrometry.

In other aspects, the filaments with ligands of the present invention can also be used in treatment methods. For example, in some embodiments, the filaments can be incorporated in wound care products including, but not limited to, wound closure products and wound dressings, wherein the compositions may be added to or incorporated in articles of manufacture. In some embodiments, the compositions of the invention are incorporated in a wound closure product or dressing in contact with the skin for capturing pathogens present in the surfaced area. In other embodiments, one or more antimicrobial compounds can also be incorporated (e.g., can be covalently and/or non-covalently incorporated in the filaments with ligands and/or other components of the wound closure products or dressing, which result in imparting antimicrobial properties (e.g., bacteriostatic and/or bactericidal properties).

In some embodiment, the present invention provides a method for treating a wound of a subject, the method comprising contacting the wound with a wound dressing comprising a therapeutically effective amount of the composition as described elsewhere herein. In certain embodiments, the wound dressing captures microorganisms from the wound. In certain embodiments, the wound dressing further comprises an antimicrobial compound. In certain embodiments, the antimicrobial compound can be covalently and/or non-covalently attached to the filament.

In some embodiments, the wound is on a surface of a skin area of the subject.

In certain embodiments, the microorganisms are pathogens.

In certain embodiments, the subject is a mammal. In certain embodiments, the subject is a human subject.

In other aspects, the present invention provides diagnostic methods. In some embodiments, the compositions and method can be used to identify/diagnose etiologic agents and/or diseases. For example, in some embodiments, the method is for diagnosing UTI, the method comprising analyzing a biological fluid of a subject (e.g., a urine sample or culture thereof) to identify a microorganism that may be present. In one embodiment, the analyzing comprises contacting the biological fluid of the subject (e.g., the urine sample or culture thereof) with the composition comprising the non-water imbibing, biocompatible filament; and the at least one affinity ligand, wherein the filament is functionalized with the at least one affinity ligand, thereby capturing a microorganism if present in the biological fluid. In other embodiments, the method further comprises identifying the microorganism. In one embodiment, the identifying can be performed using methods know in the art including, but not limited to, PCR.

### Kit

In yet another aspect, the invention provides a kit comprising the composition as described elsewhere herein and an instructional material for using the same.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### Materials and Methods

### Filament:

Nylon used in this study as the filament holding the affinity network, is a biocompatible material according to international standards of biocompatibility and it has been shown to be non-cytotoxic and to release an acceptable amount of leachable substances. The production and functionalization of nylon follows established chemistries that can be scaled up for industrial production. Material is inexpensive. In these studies, nylon was sourced and authenticated. In order to authenticate the reagents of the invention, HCl degradation and infra-red spectroscopy were used.

Procedure for Incorporating Dyes into Nylon:

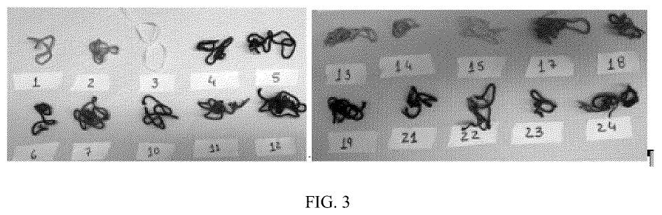

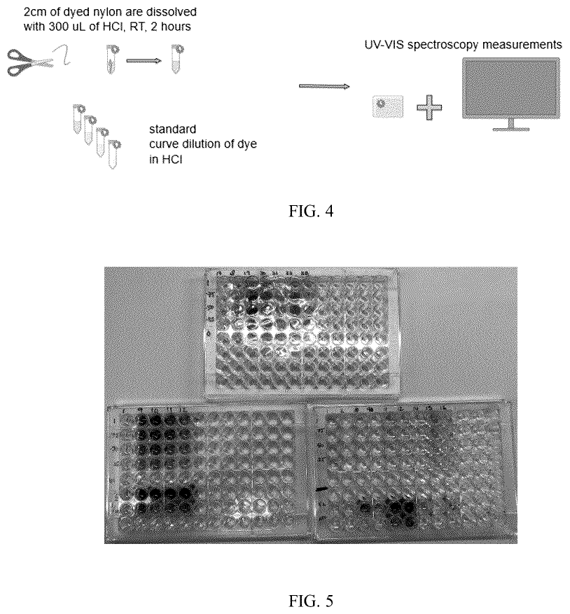

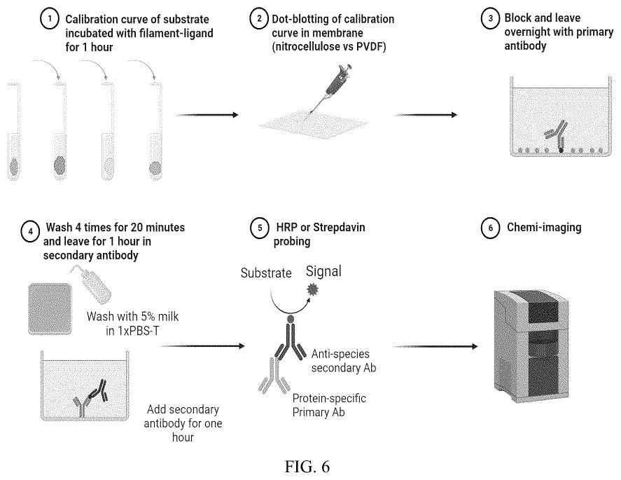

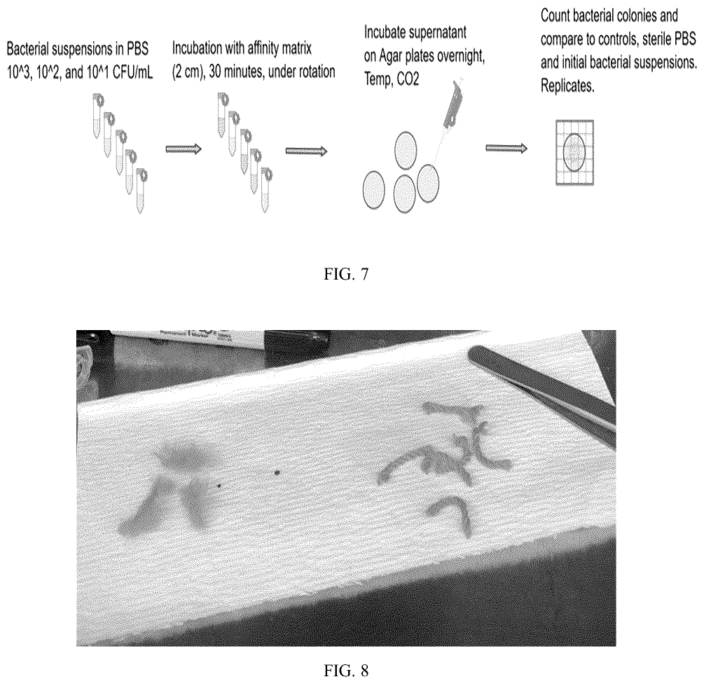

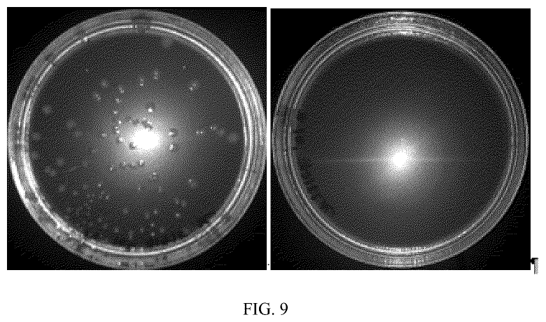

The standard procedure to incorporate dyes into the nylon is used herein. For example, about 40 centimeters of nylon thread were cut and two aliquots of 100 milligrams of dye were weighed. Nylon was incubated with an acetic acid solution at pH of 3.97. The nylon was then put into the oven and slowly heated up to a temperature of 6.5° C. for 30 minutes. The first aliquot of dye was added and incubated with nylon for 45 minutes. The temperature was then increased to 80° C., and the second aliquot of dye was added and incubated with the nylon for 60 minutes or until the temperature of the oven reached 100° C. The nylon was then washed with water multiple times and dried overnight at the room temperature. FIG. 3 shows the picture of stained nylon threads.

### Assessing Yield of Dye Incorporation:

In order to assess the yield of dye incorporation, a procedure was established to quantify the dye bound to the nylon. In order to do so, 2 centimeters of dyed nylon was completely dissolved using Hydrochloric acid (2 hours at room temperature). Subsequently, standard curves of dye dissolved in appropriate solvent (Hydrochloric acid diluted in deionized water, Methanol, DMSO, and acetonitrile) were prepared. Dissolved nylon was transferred to 96 well plates and read using a UV-VIS spectrophotometer at optimal wavelengths. FIG. 5 shows an example of a 96 well plate in which dyed nylon was dissolved.

### Procedure to Study Binding Mechanism:

In order to study the binding mechanism, a procedure was established to observe an intensity of signal in the presence of glycocalyx in supernatant. First a calibration curve was developed for LPS and LTA using the initial concentration of the purchased product as baseline. LTA was dissolved in Limulus Amebocyte Lysate (LAL) reagent water and LPS in 1×DPBS. 2 μl was then added in each dot of the calibration curve in a PVDF and Nitrocellulose membrane to verify which had a better binding for signal detection. For LTA, the nitrocellulose showed better signal detection, while for LPS, PVDF outperformed. The membrane was then blocked for 1 hour, and left in the primary antibody overnight.

The next morning, the membrane was washed with 1×PBS-T four time for 5 minutes each, and was left for one hour in the secondary antibody. The membrane was washed again using the same method as before, and developed signal (HRP or Streptavidin). The membrane was then imaged.

For the LPS and LTA inoculated with nylon, the same procedure was performed. For Gram-positive purified LTA from Staphylococcus aureus (InvivoGen Cat #tlrl-pslta), and for Gram-negative purified LPS from Escherichia coli (ThermoFisher Cat #00-4976-93) were used.

For a sandwich ELISA assay, Anti-Staphylococcus aureus LTA antibody mouse monoclonal (Sigma-Aldrich Cat #SAB4200883) and Anti-Escherichia coli LPS antibody mouse monoclonal (2D7/1) (abcam Cat #ab35654) as well as an IgG anti-mouse secondary antibody (Invitrogen Cat #62-6520) were used in this experiment to confirm the binding. Chemiluminescent Imaging (Azure C300 Biosystems) was performed to develop the dots, therefore the results of this experiment.

Screening Affinity Network for their Ability to Capture Bacteria:

Nylon affinity was functionally tested through the use of agar plates, by plating the supernatant and counting its colonies (i.e., the bacteria not captured). Affinity network were screened for their ability to capture bacteria in solution using bacteria culture plates. Nylon was reduced to an unwoven cloud and autoclaved. E. coli or S. epidermidis bacterial suspensions at 10, 100 CFU/ml in PBS were incubated with affinity networks for 60 minutes at room temperature in a rotation. Unbound fraction was mechanically separated and plated in nutrient agar plates. Bacterial cultures were held under 37° C. overnight. Colonies were manually counted and recorded.

### Example 2

Ligands were selected based on their physical chemical characteristics; nylon filaments were sourced and authenticated; dyes were incorporated into nylon filaments; and yields of incorporation were determined. After that, the nylon affinity networks were screened for their ability to bind bacteria from the solution.

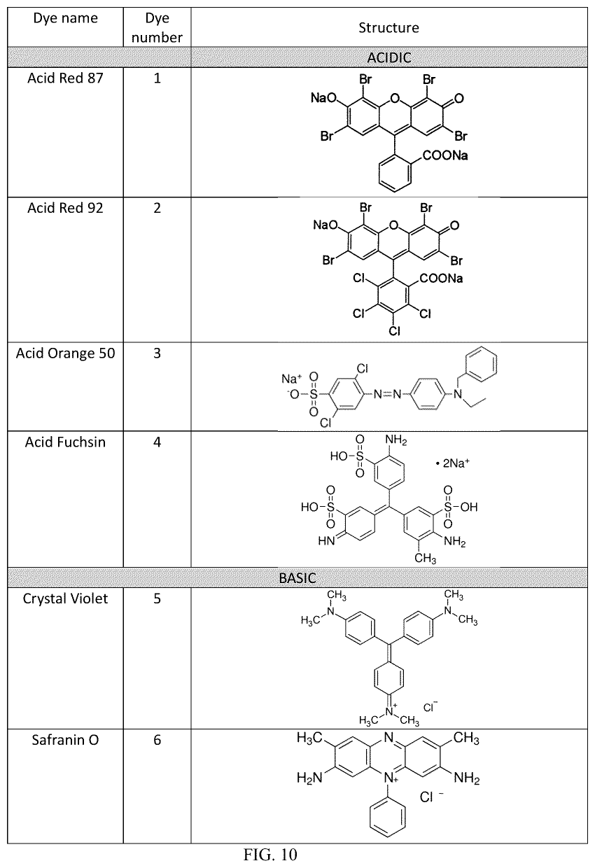

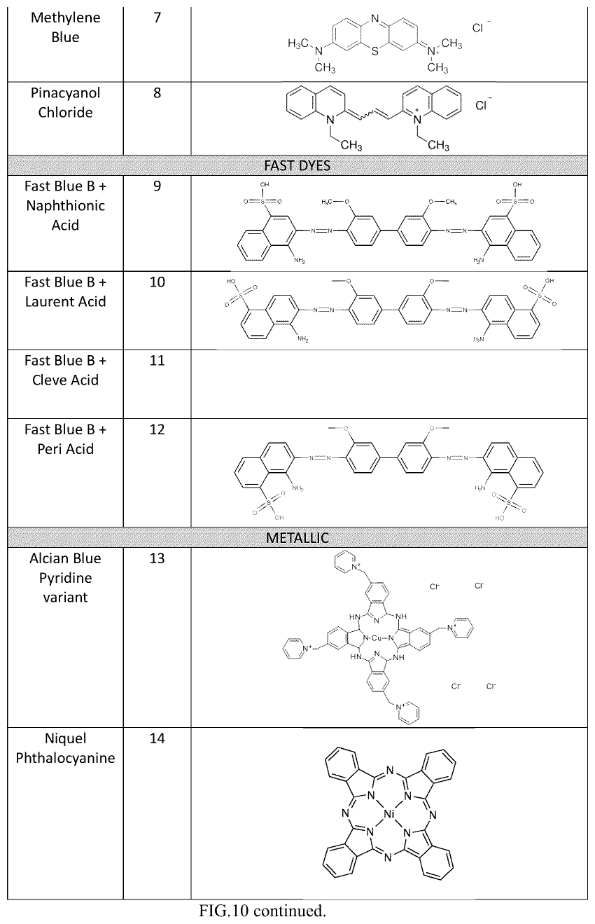

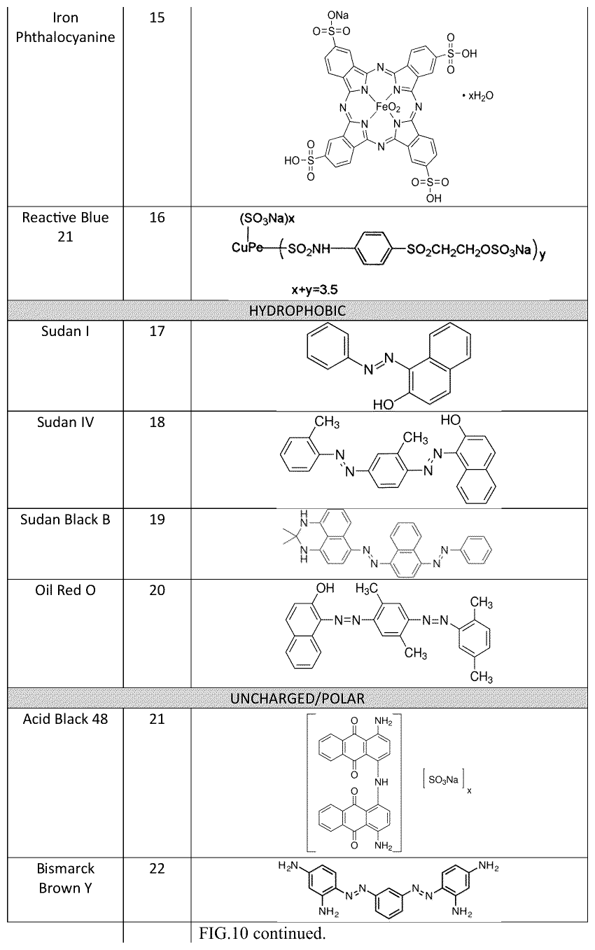

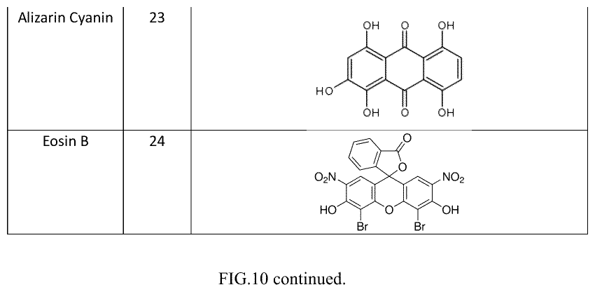

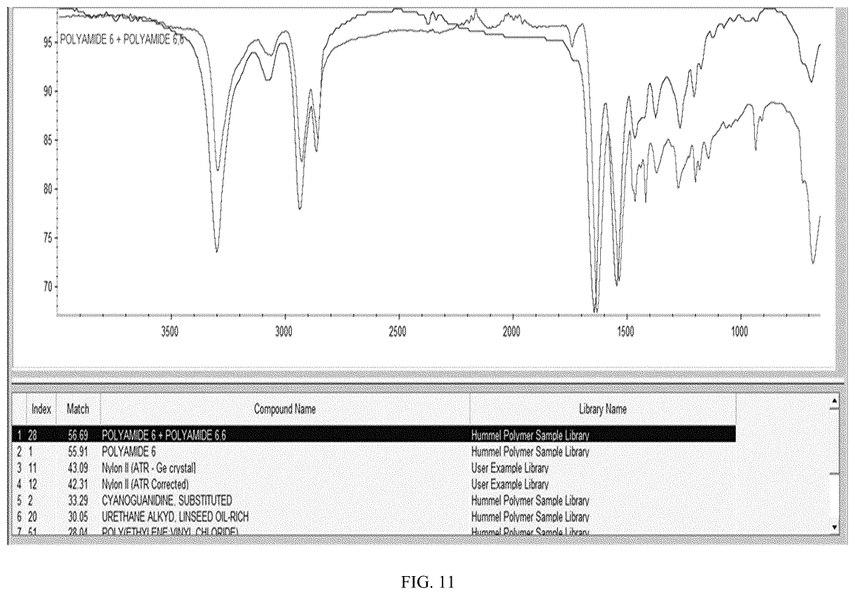

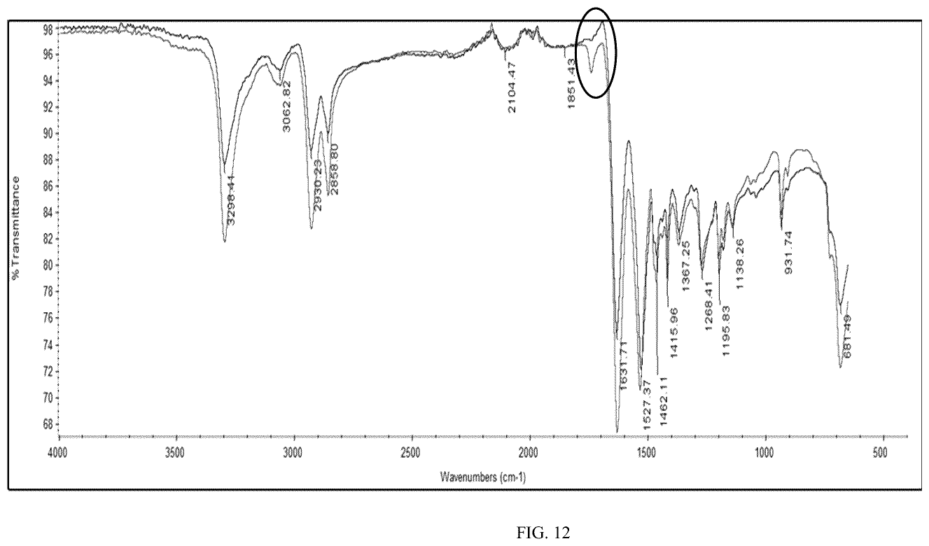

6 classes of dyes, including acidic, basic, fast dyes, metallic, hydrophobic and uncharged polar were identified. And the dyes are shown in FIG. 10.

To authenticate the nylon used in this experiment, the nylon was degraded in 37% hydrochloric acid, and subjected to a FT-IR analysis. The reading was then compared with the commercial sample spectrum, which matched with polyamide 6+ polyamide 6.6 (Hummel Polymer Sample Library OMNIC software). It was then confirmed that the nylon is indeed pure nylon, and not a mix of different polymers. Next it was sought to understand better how the dye interacts with nylon during the dying process.

A comparison of the FT-IR spectrum of an undyed nylon with a nylon dyed with methylene blue revealed a change in the N—H and C═O stretch, which according to literature, confirms the potential of an intramolecular hydrogen bond between the methylene blue dye and the nylon filament.

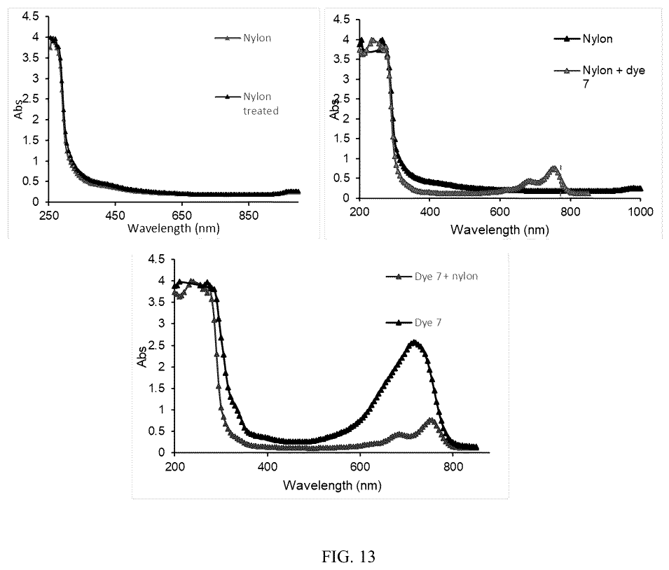

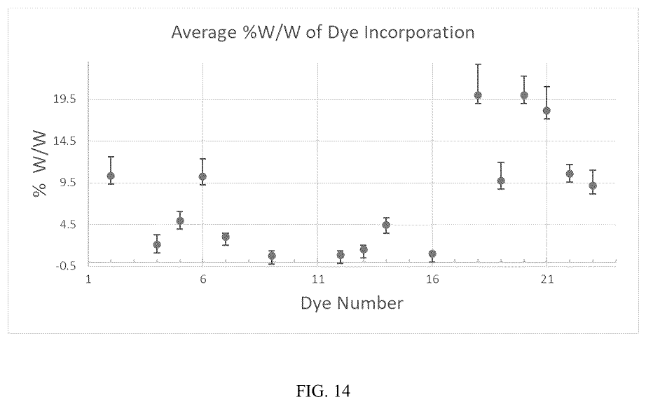

With this information it was decided to obtain an UV-VIS spectroscopy of the nylon and nylon treated with acetic acid, plus dyed nylon with methylene blue and methylene blue alone (FIG. 13). This information was used to compare the spectral scan of the nylon before and after dyeing with methylene blue to understand the molecular interaction between the dye and the nylon. Two peaks, 680 nm, and 750 nm, are present due to the addition of methylene blue FIG. 13). In FIG. 13, when the spectral scan of nylon dyed with methylene blue and methylene blue alone were compared under the same solvent and temperature conditions, the intensity of the peaks increased with a shift in the λmax. Generally, as the conjugation increases, molecules become more stable with a consequent energy decrease, which causes λmax to increase by shifting towards the right of the spectrum (redshift), and the peak intensity to increase. On the basis of this evidence, we hypothesize that the interaction between methylene blue and nylon has a heavy hydrogen bond component. It has been proved that sulfur dyes bind to nylon through hydrogen bond interactions, dispersion forces, and polar van der Waals interactions with non-site specificity. In addition, hydrogen bonds can induce bathochromic shifts, shifting the absorption towards higher wavelengths while the dipole increases the light absorbed.

Once this data was obtained, a QC/QA control method was developed so as to maintain reproducibility throughout the experiments. For this, nylons were dyed with the different dyes, and then were dissociated in HCl. The absorbance using UV-VIS spectroscopy, the dye weight is then estimated and divided by the weight of the nylon, here expressed in percentage. From then on, all of the dyed nylon must stay above the minimum range obtained through these experiments. For example, for dye 6, it must have a % W/W of at least 9% for it to be used.

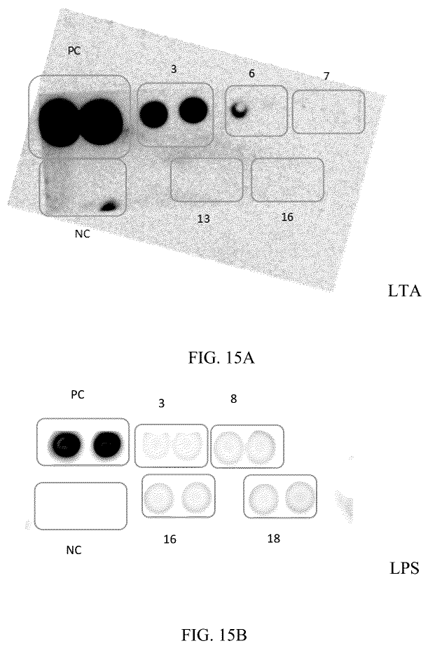

For the purpose of understanding the binding mechanism between ligand and bacteria, it was decided to do a dot-blot assay using the glycocalyx present in the Gram-positive (LTA) and Gram-negative (LPS) bacteria. It was confirmed that indeed the ligand binds to the glycocalyx of the bacterium. In FIGS. 15A-15B, the pairs of dots represent one sample incubated in a specific filament with ligand. Compared to the positive control (PC) the amount of LPS and LTA that remained in solution after incubation with the affinity net was greatly reduced (>90% reduction for most affinity ligands) This further indicates that this is most likely the mechanism of binding between the ligand and the bacteria, as the result was observed in both Gram-positive and Gram-negative.

### Example 3

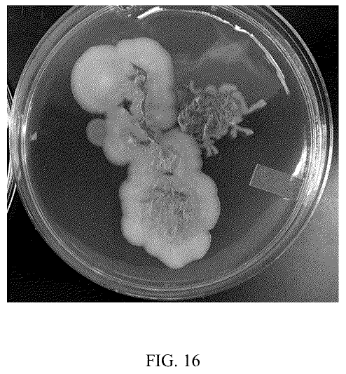

In one of the first experiments done, it was seen that the dyed nylon, for example, dyed with Safranin O, does not kill the bacteria, in fact, it was seen that the bacteria were growing from the nylon threads. This, as seen in FIG. 16, confirmed that the affinity networks are indeed capturing the bacteria, removing it from solution, and not killing them.

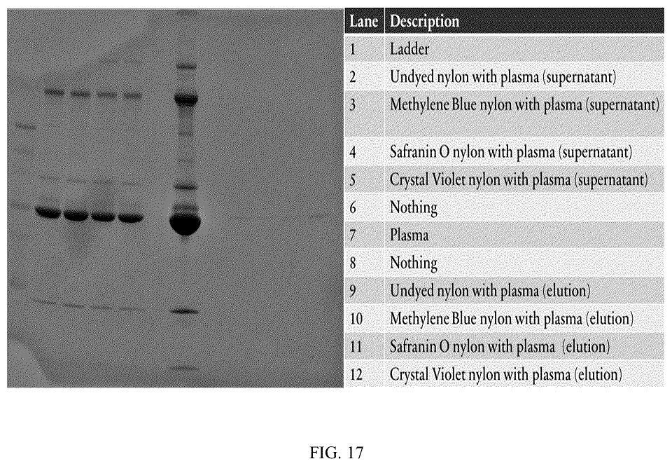

In order to assess non-specific binding to plasma proteins, Nylon networks functionalized with different basic dyes were incubated with plasma and the extracted proteins were run on an SDS PAGE gel shown in FIG. 17. Lane 7 contains 0.5 μl of plasma, Lane 2-5 contains the unbound proteins and lanes 9-12 contain the affinity net bound proteins. There is minimal non-specific binding of plasma proteins to affinity networks apart from low traces of albumin, which is present in exceedingly high quantities in the plasma proteome. This is relevant preliminary data supporting the use of this affinity networks in plasma and whole blood.

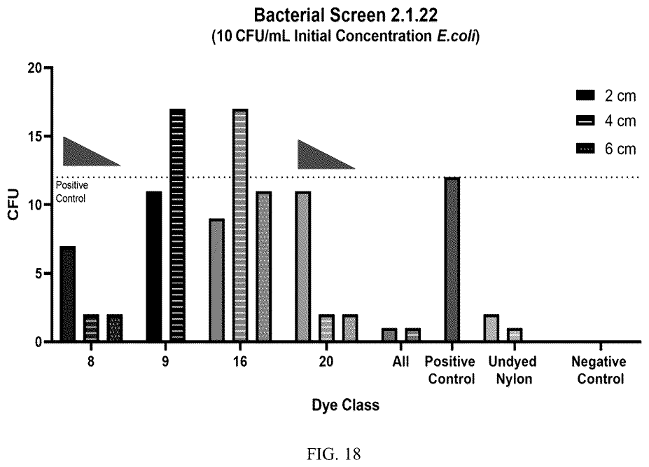

In order to establish the optimal affinity network quantity to achieve maximum bacterium capture, solutions of 10 CFU/ml of E. coli in pbs were incubated with three quantities of nylon cloud quantified in milligrams. The graph has kind of dyes in the x-axis and CFU of unbound in the y axis. Dye number 8 and 20 show a dose dependence and reach plateau at 80 milligrams. Dye 9 and 16 do not show dose dependence and it probably due to a low affinity and saturation of binding sites. Undyed nylon is confirmed to capture bacteria with high efficiency. Conclusion is that the optimal quantity of affinity net is 80 mg (FIG. 18).

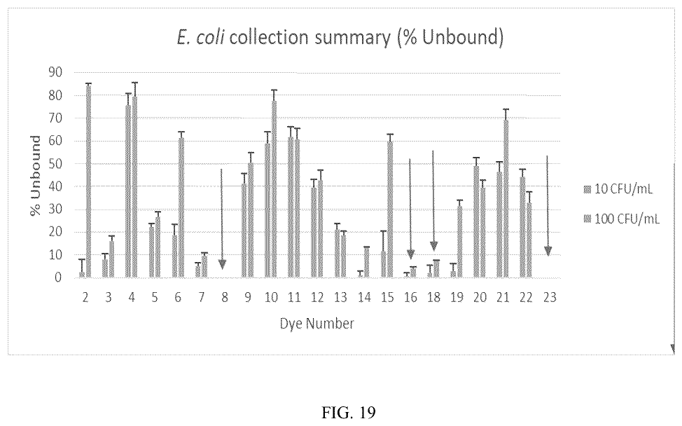

Different affinity nets were incubated with 1 mL E. coli suspension in PBS. Table. 1 (below) summarizes average CFU counts of unbound bacteria for different affinity nets. The arrows in FIG. 19 show the dyes that outperformed by having a complete or close to complete capture of bacteria in both 10 and 100 CFU/mL. These results show that some dyes outperform others in regards to capture ability, and few show an extremely high capturing yield consistent across bacteria concentration.

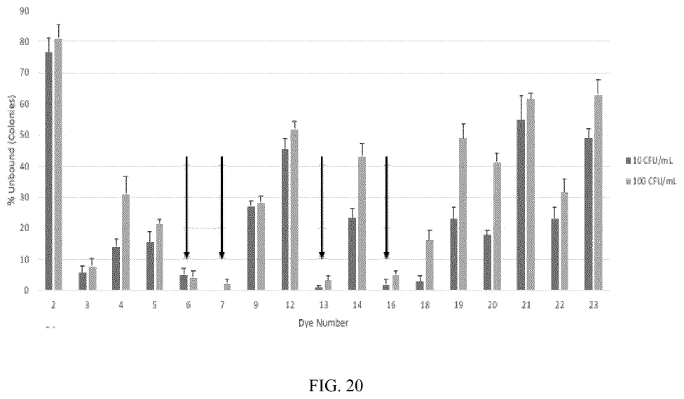

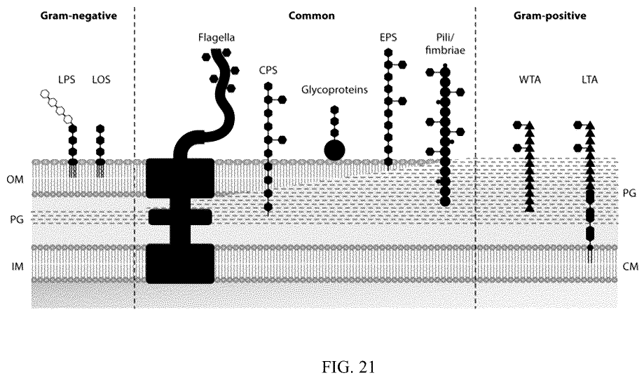

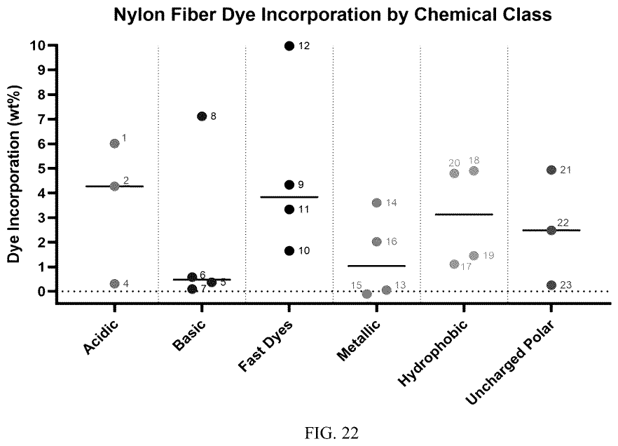

The same was done with S. epidermidis. Again, the arrows in FIG. 20 show the dyes that outperformed, with complete of close to complete capture of bacteria in both 10 and 100 CFU/mL. These results show that some dyes outperform others in regards to capture ability, and few show an extremely high capturing yield consistent across bacteria concentration.

TABLE 1
Percentage of unbound CFUs after incubation with the affinity
net (E. coli). Mean of at least 6 experiments.

| | CFU = 10 | CFU = 100 |
|---|---|---|
| NC | 0 | 0 |
| PC | 100 | 100 |
| Dye 1 | 54 | 86 |
| Dye 10 | 59 | 77 |
| Dye 11 | 62 | 60 |
| Dye 12 | 41 | 52 |
| Dye 13 | 71 | 66 |
| Dye 14 | 2 | 1 |
| Dye 15 | 12 | 60 |
| Dye 16 | 3 | 5 |
| Dye 17 | 72 | 44 |
| Dye 18 | 2 | 9 |
| Dye 19 | 3 | 31 |
| Dye 2 | 3 | 43 |
| Dye 21 | 56 | 67 |
| Dye 22 | 88 | 47 |
| Dye 23 | 0 | 0 |
| Dye 24 | 31 | 5 |
| Dye 3 | 0 | 20 |
| Dye 4 | 126 | 26 |
| Dye 5 | 0 | 0 |
| Dye 6 | 28 | 47 |
| Dye 7 | 0 | 0 |
| Dye 8 | 0 | 0 |

### Example 4

24 dyes were identified for their structural characteristics and previous knowledge of mechanisms of dye-biomolecule interaction. Dyes were incorporated into nylon affinity networks. A procedure of dye incorporation was established. Affinity nets were screened for the ability to capture bacteria spiked in PBS. Promising ligands that afforded complete or close to complete removal of bacteria from solution was observed with pinacyanol chloride, reactive blue 21, sudan IV, and alizarin cyanin as the chosen dyes for E. coli, and safranin O, methylene blue, alcian blue pyridine variant, and reactive blue 21 as the chosen dyes for S. epidermidis.

For its cytotoxic characteristics, further studies are performed, but on a basic level, it is considered that the cells are not killed, as it was demonstrated that bacteria grow in plates upon getting captured by the filaments with the ligands. Hemocompatibility tests including hemolysis, coagulation, platelet count/functions, and thrombogenicity are performed according to ISO 10993-4 to ensure safety for interactions with blood.

### Example 5

Screening to more ligands and pathogen types including bacteria B. cereus, Y. enterocolitica, S. aureus, P. aeruginosa, S. marcescens, C. acnes, Streptococcus pneumoniae, Streptococcus bovis, Treponema pallidum, Brucella sp., Leptospira interrogans, Serratia sp., Enterobacter sp., Salmonella sp., Listeria monocytogenes, Shigella spp, Neisseria meningitidis, Neisseria gonorrheae, viruses and fungi is performed. The screening is performed on plasma and blood components, and the benefits of increasing surface and binding capacity of dyes to nylon, in case of competition in a biological matrix, is investigated. Tests related to screening ligands for their cytotoxic properties, including allergic reactions, or any type of cellular inflammatory response, is also performed.

### Enumerated Embodiments

Embodiment 1 provides a composition comprising:
a non-water imbibing, biocompatible filament; and
at least one affinity ligand,
wherein the filament is functionalized with the at least one affinity ligand

Embodiment 2 provides the composition of embodiment 1, wherein the at least one affinity ligand is a dye.

Embodiment 3 provides the composition of embodiments 1-2, wherein the filament is a nylon filament.

Embodiment 4 provides the composition of embodiments 1-3, wherein the nylon filament is at least one selected from the group consisting of nylon 6; nylon 6,6; nylon 4,6; nylon 6,9; nylon 6,10; nylon 6,12; nylon 11; and nylon 12.

Embodiment 5 provides the composition of embodiments 1-4, wherein the dye is at least one selected from the group consisting of a direct dye, a sulfur dye, a reactive dye, a fast dye, an insoluble azoic dye, a phthalocyanine dye, an acid dye, a metal-complex dye, a basic dye, a metallic dye, a hydrophobic dye, an uncharged polar dye, and a disperse dye.

Embodiment 6 provides the composition of embodiments 1-5, wherein the dye is selected from the group consisting of Acid Red 87, Acid Red 92, Acid Orange 50, Acid Fuchsin, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Reactive Blue 21, Sudan I, Sudan IV, Sudan Black B, Oil Red O, Acid Black 48, Bismarck Brown Y, Alizarin Cyanin, and Eosin B.

Embodiment 7 provides the composition of embodiments 1-6, wherein the at least one affinity ligand has an affinity for a biomolecule at a surface of a microorganism.

Embodiment 8 provides the composition of embodiments 1-7, wherein the microorganism is selected from the group consisting of a bacterium, a virus, a fungus, and a protozoan.

Embodiment 9 provides the composition of embodiments 1-8, wherein the microorganism is selected from a group consisting of E. coli, B. cereus, Y. enterocolitica, S. epidermidis, S. aureus. P. aeruginosa, S. marcescens, C. acnes, Streptococcus pneumoniae, Streptococcus bovis, Treponema pallidum, Brucella sp., Leptospira interrogans, Serratia sp., Enterobacter sp., Salmonella sp., Listeria monocytogenes, Shigella spp, Neisseria meningitidis, and Neisseria gonorrheae.

Embodiment 10 provides the composition of embodiments 1-9, wherein the biomolecule is selected from the group consisting of a protein, a glycan, a glycoprotein, a lipid, and a nucleic acid.

Embodiment 11 provides an affinity matrix comprising the composition of embodiments 1-10.

Embodiment 12 provides a filtration device comprising: a column, and the affinity matrix of embodiment 11.

Embodiment 13 provides the device of embodiment 12, wherein the matrix is contained inside the column.

Embodiment 14 provides the device of embodiments 12-13, wherein the matrix is attached to the column.

Embodiment 15 provides the device of embodiments 12-14, wherein the matrix is not attached to the column.

Embodiment 16 provides the device of embodiments 12-15, wherein the microorganism is selected from the group consisting of a bacterium, a virus, a fungus, and a protozoan.

Embodiment 17 provides the device of embodiments 12-16, wherein the at least one affinity ligand is a dye.

Embodiment 18 provides a method for filtering a fluid, the method comprising: contacting the fluid with the affinity matrix of the filtration device of claim 12, wherein a microorganism, if present in the fluid, is captured by the at least one affinity ligand.

Embodiment 19 provides the method of embodiment 18, wherein the microbial population comprises at least one selected from the group consisting of a bacterium, a virus, a fungus, and a protozoan.

Embodiment 20 provides the method of embodiments 18-19 wherein the microorganism is selected from a group consisting of E. coli, B. cereus, Y. enterocolitica, S. epidermidis, S. aureus, P. aeruginosa, S. marcescens, C. acnes, Streptococcus pneumoniae, Streptococcus bovis, Treponema pallidum, Brucella sp., Leptospira interrogans, Serratia sp., Enterobacter sp., Salmonella sp., Listeria monocytogenes, Shigella spp, Neisseria meningitidis, and Neisseria gonorrheae.

Embodiment 21 provides the method of embodiments 18-20, wherein the fluid is a biological fluid or an environmental fluid.

Embodiment 22 provides the method of embodiments 18-21, wherein the biological fluid is selected from the group consisting of a blood, serum, cerebrospinal fluid, a vaginal fluid, a semen.

Embodiment 23 provides the method of embodiments 18-22, wherein the environmental fluid is one selected from the group consisting of a water, a plant lymph, and a sewerage.

Embodiment 24 provides a method for treating a wound in a subject, wherein the method comprises contacting the wound with a wound dressing comprising a therapeutically effective amount of the composition of claim 1.

Embodiment 25 provides the method of embodiment 24, wherein the method further comprises an antimicrobial compound.

Embodiment 26 provides a kit comprising the composition of claim 1 and an instructional material for using the composition.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

We claim:

1. A composition comprising:
a non-water imbibing, biocompatible filament; and
at least one affinity ligand,
wherein the filament is functionalized with the at least one affinity ligand.

2. The composition of claim 1, wherein the at least one affinity ligand is a dye.

3. The composition of claim 1, wherein the filament is a nylon filament.

4. The composition of claim 3, wherein the nylon filament is at least one selected from the group consisting of nylon 6; nylon 6,6; nylon 4,6; nylon 6,9; nylon 6,10; nylon 6,12; nylon 11; and nylon 12.

5. The composition of claim 2, wherein the dye is at least one selected from the group consisting of a direct dye, a sulfur dye, a reactive dye, a fast dye, an insoluble azoic dye, a phthalocyanine dye, an acid dye, a metal-complex dye, a basic dye, a metallic dye, a hydrophobic dye, an uncharged polar dye, and a disperse dye.

6. The composition of claim 2, wherein the dye is selected from the group consisting of Acid Red 87, Acid Red 92, Acid Orange 50, Acid Fuchsin, Crystal Violet, Safranin O, Methylene Blue, Pinacyanol Chloride, Fast Blue B+Naphthionic acid, Fast Blue B+Laurent Acid, Fast Blue B+Cleve Acid, Fast Blue B+Peri Acid, Alcian Blue Pyridine variant, Ni Phthalocyanine, Fe Phthalocyanine, Reactive Blue 21, Sudan I, Sudan IV, Sudan Black B, Oil Red O, Acid Black 48, Bismarck Brown Y, Alizarin Cyanin, and Eosin B.

7. The composition of claim 1, wherein the at least one affinity ligand has an affinity for a biomolecule at a surface of a microorganism.

8. The composition of claim 7, wherein the microorganism is selected from the group consisting of a bacterium, a virus, a fungus, and a protozoan.

9. The composition of claim 8, wherein the microorganism is selected from a group consisting of E. coli, B. cereus, Y. enterocolitica, S. epidermidis, S. aureus, P. aeruginosa, S. marcescens, C. acnes, Streptococcus pneumoniae, Streptococcus bovis, Treponema Brucella sp., Leptospira interrogans, Serratia sp., Enterobacter sp., Salmonella sp., Listeria monocytogenes, Shigella spp, Neisseria meningitidis, and Neisseria gonorrheae.

10. The composition of claim 7, wherein the biomolecule is selected from the group consisting of a protein, a glycan, a glycoprotein, a lipid, and a nucleic acid.

11. An affinity matrix comprising the composition of claim 1.

12. A filtration device comprising:
a column, and the affinity matrix of claim 11.

13. The device of claim 12, wherein the matrix is contained inside the column.

14. The device of claim 12, wherein the matrix is attached to the column.

15. The device of claim 12, wherein the matrix is not attached to the column.

16. The device of claim 12, wherein the microorganism is selected from the group consisting of a bacterium, a virus, a fungus, and a protozoan.

17. The device of claim 12, wherein the at least one affinity ligand is a dye.

18. A method for filtering a fluid, the method comprising:
contacting the fluid with the affinity matrix of the filtration device of claim 12, wherein a microorganism, if present in the fluid, is captured by the at least one affinity ligand.

19. The method of claim 18, wherein the microorganism comprises at least one microbe selected from the group consisting of a bacterium, a virus, a fungus, and a protozoan.

20. The method of 19, wherein the at least one microbe is selected from a group consisting of E. coli, B. cereus, Y. enterocolitica, S. epidermidis, S. aureus, P. aeruginosa, S. marcescens, C. acnes, Streptococcus pneumoniae, Streptococcus bovis, Treponema pallidum, Brucella sp., Leptospira interrogans, Serratia sp., Enterobacter sp., Salmonella sp., Listeria monocytogenes, Shigella spp, Neisseria meningitidis, and Neisseria gonorrheae.

21. The method of claim 18, wherein the fluid is a biological fluid or an environmental fluid.

22. The method of claim 21, wherein the biological fluid is selected from the group consisting of a blood, serum, cerebrospinal fluid, a vaginal fluid, and a semen,

23. The method of claim 21, wherein the environmental fluid is one selected from the group consisting of water, a plant lymph, and a sewerage.

24. A method for treating a wound, the method comprising contacting the wound with a wound dressing comprising a therapeutically effective amount of the composition of claim 1.

25. The method of claim 24, wherein the method further comprises an antimicrobial compound.

26. A kit comprising the composition of claim 1 and an instructional material for using the composition.
